(19) United States
(12) Patent Application Publication
(10) Pub. No.: US 2026/0081037 A1
(43) Pub. Date: Mar. 19, 2026

(54) METHODS AND SYSTEMS FOR PREDICTING INTENSIVE CARE UNIT MORTALITY

(71) Applicant: KONINKLIJKE PHILIPS N.V., EINDHOVEN (NL)

(72) Inventors: LOUIS NICOLAS ATALLAH, BOSTON, MA (US); MOHSEN NABIAN, BOSTON, MA (US); EMMA HOLDRICH SCHWAGER, DENVER, CO (US); OMAR BADAWI, BALTIMORE, MD (US); XINGGANG LIU, BALTIMORE, MD (US); PAMELA JAYNE AMELUNG, BALTIMORE, MD (US)

(21) Appl. No.: 19/110,508

(22) PCT Filed: Sep. 6, 2023
(86) PCT No.: PCT/EP2023/074354

§ 371 Date: Mar. 11, 2025

Related U.S. Application Data
(60) Provisional application No. 63/406,462, filed on Sep. 14, 2022.

(51) Int. Cl. G16H 50/50; G16H 10/60

(52) U.S. Cl. CPC G16H 50/50; G16H 10/60

(57) ABSTRACT

The present disclosure relates to methods and systems for predicting intensive care unit (ICU) mortality. More specifically, the methods and systems for predicting a likelihood of ICU mortality described herein enable robust modeling of ICU mortality that addresses biases in automated data collection, including variations in documentation practices across different units, different hospital systems, and across time. In certain embodiments, the methods described herein include: providing an ICU mortality prediction system; obtaining a plurality of records for a patient in an ICU covering at least a first time period; extracting a plurality of different defined ICU prediction features for the patient; analyzing the extracted plurality of different defined ICU prediction features using a trained ICU mortality prediction model; generating a likelihood ICU mortality for the patient based on the analysis; and presenting the generated likelihood of ICU mortality for the patient via a user interface.

### FIELD OF THE DISCLOSURE

The present disclosure relates generally to methods and systems for predicting intensive care unit (ICU) mortality, and more specifically to methods and systems for predicting intensive care unit mortality using risk adjusted models.

### BACKGROUND

Widespread adoption of electronic health records has enabled automated data capturing and propelled predictive risk modeling in a variety of respects and across many different cohorts. In certain settings, risk adjusted predictive modeling has become an essential pillar for measuring outcomes and other benchmarking.

For example, several severity scores exist to measure intensive care unit (ICU) performance, but several issues limit their utility for benchmarking performance across ICUs and over time. In particular, risk models are increasingly calculated through automated, direct extraction of electronic health record (EHR) data, which solves many issues of efficiency and inter-rater reliability, but also introduces new risks of bias through variation in documentation patterns (e.g., measurement error and/or data drift, etc.). When these variations are non-random and correlated with institutions, significant bias can be introduced, artificially improving or worsening measured performance for an institution relative to its peers.

In some cases, the sources of bias may be observed, such as when certain ICUs do not chart a particular condition or status. In other cases, the sources of bias may be harder to observe but still impact performance. For example, the primary reason for admission to an ICU can have a significant impact on mortality prediction, but the choice of diagnosis can be highly subjective and vary across institutions and over time. As a result, these variations can have a tangible effect on risk estimates.

### SUMMARY OF THE DISCLOSURE

Accordingly, there is a continued need for clinical systems that more accurately predict patient mortality while in the ICU, including whether the patient is likely to be discharged alive or deceased.

According to an embodiment of the present disclosure, a method for predicting a likelihood of intensive care unit (ICU) mortality for a patient using an ICU mortality prediction system is provided. The method comprises: providing an ICU mortality prediction system; obtaining, from an electronic medical records database, a plurality of records for a patient in an ICU covering at least a first time period; extracting, from the obtained plurality of records, a plurality of different defined ICU prediction features for the patient; analyzing the extracted plurality of different defined ICU prediction features using a trained ICU mortality prediction model; generating, from the analysis, a likelihood of ICU mortality for the patient; and presenting, via a user interface of the ICU mortality prediction system, the generated likelihood of ICU mortality for the patient. The ICU mortality prediction model can be trained, validated and tested by: obtaining, from an electronic medical records database, a plurality of records for each of a plurality of patients in an ICU covering at least a first time period; extracting, from the obtained plurality of records, a plurality of different health features for each of the plurality of patients; manually curating, based on the results of extracting, the extracted plurality of different health features to identify the plurality of different defined ICU prediction features, wherein the manual curation is configured to minimize outlier bias, and wherein admission diagnosis is one of the plurality of different defined ICU prediction features and further wherein manual curation comprises grouping admission diagnoses into one or more groups using clinical knowledge to minimize misclassification; training the ICU mortality prediction model using the plurality of different defined ICU prediction features for at least some of the plurality of patients; and storing the trained ICU mortality prediction model.

In an aspect, the first time period is at least 24 hours in the ICU.

In an aspect, the extracted plurality of different defined ICU prediction features for the subject comprises some or all of the features in Table 1.

In an aspect, the likelihood of ICU mortality for the patient further comprises a mortality timeline.

In an aspect, the ICU mortality prediction system is, or is a component of, a patient data management systems (PDMS) or a patient monitoring system.

In an aspect, the patient is a historical patient.

In an aspect, the patient is in the ICU during the presentation of the generated likelihood of ICU mortality.

In an aspect, the trained ICU mortality prediction model can analyze the extracted plurality of different defined ICU prediction features and generate a likelihood of ICU mortality for the patient when some of the plurality of different defined ICU prediction features are missing from the obtained plurality of records.

In an aspect, the extracted plurality of different defined ICU prediction features for the subject comprises a total Glasgow Coma Scale (GCS) score, wherein the GCS is a GCS assessed most recently during the first time period.

In an aspect, the ICU mortality prediction model is a generalized additive model (GAM).

According to another embodiment of the present disclosure, an intensive care unit (ICU) mortality prediction system configured to predict a likelihood of ICU mortality for a patient is provided. The ICU mortality prediction system comprises: an electronic medical records database comprising a plurality of records for a plurality of patients; a trained ICU mortality prediction model configured to analyze a plurality of different defined ICU prediction features to generate a likelihood of ICU mortality for a patient; a processor; and a user interface configured to provide the generated likelihood of ICU mortality for the patient. The processor can be configured to: (i) obtain, from the electronic medical records database, a plurality of records for a patient in an ICU covering at least a first time period; (ii) extract, from the obtained plurality of records, a plurality of different defined ICU prediction features for the patient; (iii) analyze the extracted plurality of different defined ICU prediction features using the trained ICU mortality prediction model; and (iv) generate, from the analysis, a likelihood of ICU mortality for the patient. The ICU mortality prediction model can be trained by: obtaining, from an electronic medical records database, a plurality of records for each of a plurality of patients in an ICU covering at least a first time period; extracting, from the obtained plurality of records, a plurality of different health features for each of the plurality of patients; manually curating, based on the results of extracting, the extracted plurality of different health features to identify the plurality of different defined ICU prediction features, wherein the manual curation is configured to minimize outlier bias, and wherein admission diagnosis is one of the plurality of different defined ICU prediction features and further wherein manual curation comprises grouping admission diagnoses into one or more groups using clinical knowledge to minimize misclassification; training the ICU mortality prediction model using the plurality of different defined ICU prediction features for at least some of the plurality of patients; and storing the trained ICU mortality prediction model.

In an aspect, the first time period is at least 24 hours in the ICU.

In an aspect, the extracted plurality of different defined ICU prediction features for the subject comprises some or all of the features in Table 1.

In an aspect, the ICU mortality prediction system is a patient data management systems (PDMS) or a patient monitoring system.

In an aspect, the ICU mortality prediction model is a generalized additive model (GAM).

These and other aspects of the various embodiments will be apparent from and elucidated with reference to the embodiments described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is a flowchart of a method for predicting a likelihood of mortality illustrated according to aspects of the present disclosure.

FIG. 2 is a schematic diagram of an ICU mortality prediction system illustrated according to aspects of the present disclosure.

FIG. 3 is a flowchart of a method for training a mortality prediction model illustrated according to aspects of the present disclosure.

FIG. 4A is a graph of the APACHE IVa ICU model performance on an extended dataset, illustrated according to aspects of the present disclosure.

FIG. 4B is a graph of the APACHE IVb ICU model performance on an extended dataset, illustrated according to aspects of the present disclosure.

FIG. 4C is a graph of an inventive IUC mortality prediction model performance, illustrated according to aspects of the present disclosure.

FIG. 5A is a graph of the APACHE IVa hospital model performance on an extended dataset, illustrated according to aspects of the present disclosure.

FIG. 5B is a graph of the APACHE IVb hospital model performance on an extended dataset, illustrated according to aspects of the present disclosure.

FIG. 5C is a graph of an inventive hospital mortality prediction model performance, illustrated according to aspects of the present disclosure.

FIG. 6 is a graph comparing actual/predicted ratios and AUC metrics for APACHE models and an inventive model, illustrated according to aspects of the present disclosure.

FIG. 7 is a graph comparing actual/predicted ratio ICU mortality metrics for different mortality prediction models across time, illustrated according to aspects of the present disclosure.

FIG. 8 is a graph comparing actual/predicted ratio hospital mortality metrics for different mortality prediction models across time, illustrated according to aspects of the present disclosure.

FIG. 9 is a graph demonstrating the effect of a GCS documentation change on model outcomes, illustrated according to aspects of the present disclosure.

FIG. 10 is another graph demonstrating the effect of a GCS documentation change on model outcomes, illustrated according to aspects of the present disclosure.

### DETAILED DESCRIPTION OF EMBODIMENTS

The present disclosure is directed to methods and systems for predicting intensive care unit mortality based on clinical features using risk models that mitigate different biases. As described herein, the methods and systems reduce the documentation burden to obtain mortality risk predictions, reduce the bias introduced through variations in documentation practice, meet and/or exceed current accuracy and performance benchmarks, and eliminate.

The embodiments and implementations disclosed or otherwise envisioned herein can be utilized with any patient care system, including but not limited to clinical decision support tools, among other systems. For example, one application of the embodiments and implementations herein is to improve analysis systems such as, e.g., the Philips® eCareManager Enterprise telehealth products, Philips® Tasy EMR solutions, and Philips® Patient Flow Capacity Suite products, among many others. However, the disclosure is not limited to these devices or systems, and thus disclosure and embodiments disclosed herein can encompass any device or system capable of generated and reporting information about an ICU stay for a patient.

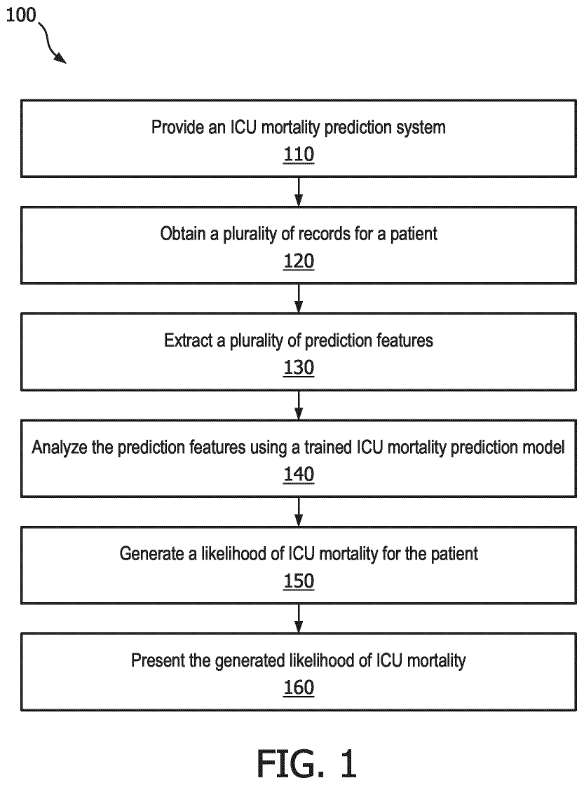

Turning to FIG. 1, a flowchart of a method 100 for predicting a likelihood of intensive care unit (ICU) mortality for a patient using an ICU mortality prediction system is illustrated according to aspects of the present disclosure. The ICU mortality prediction system can be any of the systems described or otherwise envisioned herein.

At a step 110 of the method 100, according to an embodiment, an ICU mortality prediction system 200 is provided. As discussed in greater detail below, the ICU mortality prediction system 200 can be configured predict a likelihood of ICU mortality for a patient. In embodiments, the ICU mortality prediction system 200 can include a trained ICU mortality prediction model 264 configured to analyze a plurality of different defined ICU prediction features to generate a likelihood of ICU mortality for the patient. In some embodiments, one or more ICU mortality prediction model(s) 264 may be developed using a generalized additive model (GAM) framework, which allows the ICU mortality prediction model(s) 264 to use non-linear functions of continuous features while maintaining the additivity of multivariate linear regression. However, other models are possible.

At a step 120, the method 100 includes obtaining a plurality of records for a patient in an ICU. In some embodiments, the plurality of records for the patient may be obtained by the ICU mortality prediction system 200. In further embodiments, the plurality of records for the patient may be obtained from an electronic medical records database 270A, 270B. For example, the electronic medical records database 270A, 270B may include patient unit stays admitted to ICUs where physiologic, diagnosis, and treatment information (collectively, “medical information” or “medical data”) are captured, charted, or otherwise recorded. That is, the electronic medical records database 270A, 270B can comprise a plurality of healthcare-related records for a plurality of patients, including historical patients and/or patients of current ICU stays.

In still further embodiments, the plurality of records obtained in step 120 can include medical records that cover a first period of time. For example, the plurality of records obtained in step 120 can include medical records that cover the first 24 hours of the patient's stay in an ICU (i.e., the medical data available through the first day of ICU admission), although longer and shorter periods of time are possible.

Alternatively, if medical records for the patient within the first day of ICU admission are not available, the medical records may include, for example and without limitation, medical records covering up to six hours before ICU admission, although longer and shorter time periods are possible.

As such, in various examples, the first period of time can include the first 24 hours of the patient's ICU stay, only the first 24 hours of the patient's ICU stay, less than 24 hours of the patient's ICU stay, an amount of time (e.g., 1 hour, 3 hours, 6 hours, 12 hours, etc.) preceding the patient's admission to the ICU, and/or some combination thereof.

At a step 130, the method 100 includes extracting a plurality of different defined ICU prediction features for the patient from the plurality of records obtained in step 120. In embodiments, the ICU mortality prediction system 200 may extract the plurality of different defined ICU prediction features for the patient based on the plurality of records obtained in step 120.

As used herein, the term “defined ICU prediction features” refers to continuous physiologic, diagnosis, and/or treatment information that are defined prior to analyzing the plurality of medical records of the patient using a trained model. In embodiments, the plurality of different defined ICU prediction features can include of the defined prediction features shown in Table 1 below:

TABLE 1
LIST OF ICU PREDICTION FEATURES.

| Data Input Category | Data Input | Detailed Definition |
|---|---|---|
| Basic characteristics | BMI (Body Mass Index) | Kg/m² |
| Basic characteristics | Age | Years |
| Basic characteristics | Gender | Female, non-female (or N/A) |
| Basic characteristics | Pre-ICU admission lead time | Hours in the hospital before ICU |
| Basic characteristics | Ventilation status | Yes or no, at hour 24 of ICU |
| | | admission |
| Basic characteristics | Admitted with elective | Yes or no |
| | surgery status | |
| Vital signs | Mean blood pressure | mmHg, mean, variability |
| Vital signs | Systolic blood pressure | mmHg, mean |
| Vital signs | Diastolic blood pressure | mmHg, mean |
| Vital signs | Heart rate | Rate per minute, mean, variability |
| Vital signs | Respiratory rate | Rate per minute, mean, variability |
| Vital signs | Oxygen saturation, SpO2 | %, mean |
| Labs | Blood glucose | mg/dl, mean |
| Labs | White blood cell count | Count per ml, mean |
| Labs | Blood sodium | mEq/L, mean |
| Labs | Blood potassium | mEq/L, mean |
| Labs | Blood creatinine | mEq/L, mean |
| Labs | Blood hemoglobin | g/dl, mean |
| Labs | Blood albumin | g/dl, mean, with missing |
| Labs | Blood lactate | mmol/L, mean, with missing |
| Labs | Arterial blood gas, pH | Mean, with missing |
| Labs | Arterial blood gas, PaCO2 | mmHg, mean, with missing |
| Provider assessment | Admission diagnosis | Defined category list |
| Provider assessment | Total Glasgow Coma Scale | GCS scores (3-15) with unable to |
| | score (GCS) | score due to medication, NA; last |
| | | entry at 24 hours of ICU |
| | | admission |

As shown in Table 1, the plurality of different defined ICU prediction features can include one or more different data inputs from one or more different data input categories. In some embodiments, the plurality of different defined ICU prediction features include multiple records for a data input taken over time. For example, the heart rate of the patient may be extracted over a period of time such that a mean and variability statistics can also be extracted. In other embodiments, the plurality of different defined ICU prediction features includes only a single record for a particular data input. For example, the extracted plurality of different defined ICU prediction features can include a total GCS representative of the GCS assessed most recently during the first time period.

However, the plurality of different defined ICU prediction features are not limited to only these features, and it is contemplated that other data input categories and other data inputs may be defined in future models. In particular embodiments, the prediction features may be defined to ensure a clinically accurate reflection of the patient while minimizing the impact of potentially anomalous outlier values through the use of means and measures of variability, rather than relying on the most extreme values used in conventional risk models.

In embodiments, the plurality of different defined ICU prediction features may be automatically extracted from the plurality of records obtained in step 120 using natural language processing and/or a machine learning algorithm. For example, the ICU mortality prediction system 200 may include a prediction feature extractor 261 that implements a natural language processing technique and/or a machine learning algorithm in order to extract the plurality of different predefine ICU prediction features.

At a step 140, the method 100 includes analyzing the extracted plurality of different defined ICU prediction features using a trained ICU mortality prediction model 264. In embodiments, the ICU mortality prediction system 200 can apply the trained ICU mortality prediction model(s) 264 to the extracted plurality of different defined ICU prediction features.

In embodiments, the extracted plurality of different predefine ICU prediction features may be analyzed by fitting the prediction features to the trained ICU mortality prediction model(s) 264. In some embodiments, one or more of ICU mortality prediction model(s) 264 may be a generalized additive model that allows the ICU mortality prediction model(s) 264 to use non-linear functions of continuous features while maintaining the additivity of multivariate linear regression.

As such, the trained ICU mortality prediction model(s) 264 may enable a certain degree of freedom (such as at least four degrees of freedom) between each of the extracted prediction features to allow for non-linear relationships with the outcomes. That is, the trained ICU mortality prediction model(s) 264 can include interaction terms to account for features that have a different association with the outcome(s) depending on one or more other features with which they interact.

In further embodiments, the trained ICU mortality prediction model(s) 264 may enable analysis of the extracted plurality of different defined ICU prediction features even when one or more defined prediction features are missing from the extracted prediction features for the patient. For example, in some embodiments, one or more variables that are less commonly measured at ICU admission may be missing from the patient's medical records (and therefore not included in the patient's records received in step 120). In specific embodiments, the trained ICU mortality prediction model(s) 264 may be used even though one or more of the data inputs listed in Table 1 above are missing.

At a step 150, the method 100 includes generating a likelihood of ICU mortality for the patient based on the analysis performed in step 140. In embodiments, the likelihood of ICU mortality can represent a mortality prediction within a certain amount of time for a patient admitted to an ICU (as opposed to a hospital-level likelihood of mortality). In some embodiments, the likelihood of ICU mortality can be a mortality prediction for the patient within 48 hours after discharge, within 72 hours after discharge, within 28 days, within 90 days, among others. In embodiments, the likelihood of ICU mortality generated for the patient includes a mortality timeline, which may be a series of mortality predictions for the patient calculated for multiple points in time.

At a step 160, the method 100 includes presenting the generated likelihood of ICU mortality for the patient. For example, in embodiments, the generated likelihood of ICU mortality for the patient may be presented to a healthcare worker, administrator, and/or provider responsible for the patient. In some embodiments, the generated likelihood of ICU mortality for the patient may be presented via a user interface, such as a display screen or computer monitor. In embodiments, the user interface used to present the generated likelihood of ICU mortality for the patient may be a user interface 240 of the ICU mortality prediction system 200. In still further embodiments, the patient is still admitted to the ICU while the likelihood of ICU mortality is generated and/or presented (e.g., the method 100 is performed before the patient is discharged from the ICU).

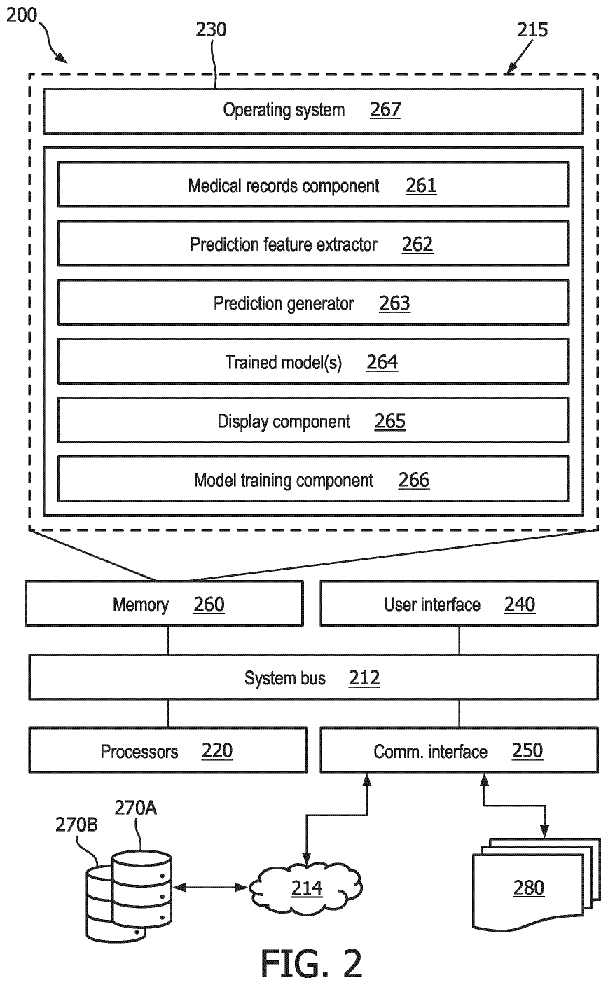

Turning to FIG. 2, an example ICU mortality prediction system 200 is illustrated. The ICU mortality prediction system 200 can be configured to predict a likelihood of ICU mortality for a patient, as described above. In some embodiments, the ICU mortality prediction system 200 may be at least part of a larger patient data management system (PDMS) and/or a patient monitoring system.

In embodiments, the ICU mortality prediction system 200 comprises one or more processors 220, machine-readable memory 260, a user interface 240, and/or a communications interface 250, all of which may be interconnected and/or communication through a system bus 212 containing conductive circuit pathways through which instructions (e.g., machine-readable signals) may travel to effectuate communication, tasks, storage, and the like.

As discussed in more detail below, the one or more processors 220 may be configured to perform one or more steps of the methods described herein, including but not limited to, the following: (i) obtain, from an electronic medical records database 270A, 270B, a plurality of records for one or more patients in an ICU covering at least a first time period; (ii) extract, from the obtained plurality of records, a plurality of different defined ICU prediction features for one or more patients; (iii) analyze the extracted plurality of different defined ICU prediction features using a trained ICU mortality prediction model 264; and (iv) generate, from the analysis, a likelihood of ICU mortality for one or more patients.

In some examples, the one or more processors 220 may include a high-speed data processor adequate to execute the program components described herein and/or various specialized processing units as may be known in the art. In some examples, the one or more processors 220 may be a single processor, multiple processors, or multiple processor cores on a single die.

In some examples, the communications interface 250 can include a network interface configured to connect the ICU mortality prediction system 200 to a communications network 214, an input/output (“I/O”) interface configured to connect and communicate with one or more peripheral devices, a memory interface configured to accept, communication, and/or connect to a number of machine-readable memory devices, and the like.

In certain embodiments, the communications interface 250 may operatively connect the ICU mortality prediction system 200 to a communications network 214, which can include a direct interconnection, the Internet, a local area network (“LAN”), a metropolitan area network (“MAN”), a wide area network (“WAN”), a wired or Ethernet connection, a wireless connection, and similar types of communications networks, including combinations thereof. In some examples, ICU mortality prediction system 200 may communicate with one or more remote/cloud-based servers (e.g., the electronic medical records database 270A), cloud-based services, and/or remote devices via the communications network 214.

The memory 260 can be variously embodied in one or more forms of machine-accessible and machine-readable memory. In some examples, the memory 260 includes a storage device that comprises one or more types of memory. For example, a storage device can include, but is not limited to, a non-transitory storage medium, a magnetic disk storage, an optical disk storage, an array of storage devices, a solid-state memory device, and the like, including combinations thereof.

Generally, the memory 260 is configured to store data/information and instructions 215 that, when executed by the one or more processors 220, causes the ICU mortality prediction system 200 to perform one or more tasks. In particular examples, the memory 260 includes an ICU mortality prediction package 230 that causes the ICU mortality prediction system 200 to perform one or more steps of the methods described herein.

In embodiments, the ICU mortality prediction package 230 comprises a collection of program components, database components, and/or data. Depending on the particular implementation, the ICU mortality prediction package 230 may include software components, hardware components, and/or some combination of both hardware and software components.

The ICU mortality prediction package 230 may include one or more software packages configured to predict a likelihood of ICU mortality for a patient. These software packages may be incorporated into, loaded from, loaded onto, or otherwise operatively available to and from the ICU mortality prediction system 200.

In some examples, the ICU mortality prediction package 230 and/or one or more individual software packages may be stored in a local storage device 260. In other examples, the ICU mortality prediction package 230 and/or one or more individual software packages may be loaded onto and/or updated from a remote server via the communications interface 250.

In particular embodiments, the ICU mortality prediction package 230 can include, but is not limited to, instructions 215 having a medical records component 261, prediction feature extractor 262, a prediction generator 263, one or more trained ICU mortality prediction models 264, a display component 263, and/or a model training component 266. These components may be incorporated into, loaded from, loaded onto, or otherwise operatively available to and from the ICU mortality prediction system 200.

In embodiments, the medical records component 260 can be a stored program component that is executed by at least one processor, such as the one or more processors 220 of the ICU mortality prediction system 200. In particular, the medical records component 260 can be configured to interface with an electronic medical records database 270A in order to obtain a plurality of records for one or more patients, as described herein. That is, the medical records component 260 may be configured to request, receive, and/or otherwise obtain a plurality of medical records for one or more patients in an ICU.

In embodiments, one or more of the patients may be historical patients. In other embodiments, one or more of the patients may be current ICU patients. In still further embodiments, the medical records component 260 may obtain a plurality of records for a combination of historical and/or current ICU patients.

In embodiments, the prediction feature extractor 261 can be a stored program component that is executed by at least one processor, such as the one or more processors 220 of the ICU mortality prediction system 200. In particular, the prediction extractor 261 can be configured to extract a plurality of different predefine ICU prediction features for a patient, as described herein. In particular, the prediction feature extractor 261 can be configured to extract predefine ICU prediction features from the plurality of records obtained from an electronic medical records database 270A using natural language processing and/or a machine learning algorithm.

In embodiments, the prediction generator 263 can be a stored program component that is executed by at least one processor, such as the one or more processors 220 of the ICU mortality prediction system 200. In particular, the prediction generator 263 can be configured to analyze the extracted plurality of different predefine ICU prediction features and generate a likelihood of ICU mortality, as described herein.

In particular embodiments, the prediction generator 263 can be configured to use one or more trained ICU mortality prediction model(s) 264 in order to analyze the extracted ICU prediction features. Based on the output of applying the one or more trained ICU mortality prediction model(s) 264, the prediction generator 263 may generate a likelihood of ICU mortality for a particular patient.

In embodiments, the display component 265 can be a stored program component that is executed by at least one processor, such as the one or more processors 220 of the ICU mortality prediction system 200. In particular, the display component 265 can be configured operate a user interface 240 in order to present the generated likelihood of ICU mortality for the patient, as described herein. In some embodiments, the display component 265 can include a programmable processor, also referred to as a graphics progressing units (GPU), which is specialized for rendering images on a monitor or display screen of a user interface 240. In other words, the user interface 240 may be configured, via a display component 265, to provide or otherwise present a likelihood of ICU mortality generated for one or more patients.

The ICU mortality prediction system 200 may also include an operating system component 267, which may be stored in the memory 260. The operating system component 267 may be an executable program facilitating the operation of the ICU mortality prediction system 200. Typically, the operating system component 267 can facilitate access of the communications interface 250, and can communicate with other components of the ICU mortality prediction system 200, including but not limited to, the user interface 240, the memory 260, and/or the electronic medical records database 270A.

According to certain embodiments, the ICU mortality prediction system 200 includes at least an electronic medical records database 270A, 270B, a processor 220, a user interface 240, and a trained ICU mortality prediction model 264. In embodiments, the ICU mortality prediction model 264 may be trained using a training dataset 280 comprising a plurality of records for each of a plurality of patients over a period of time covering each patient's stay in an ICU.

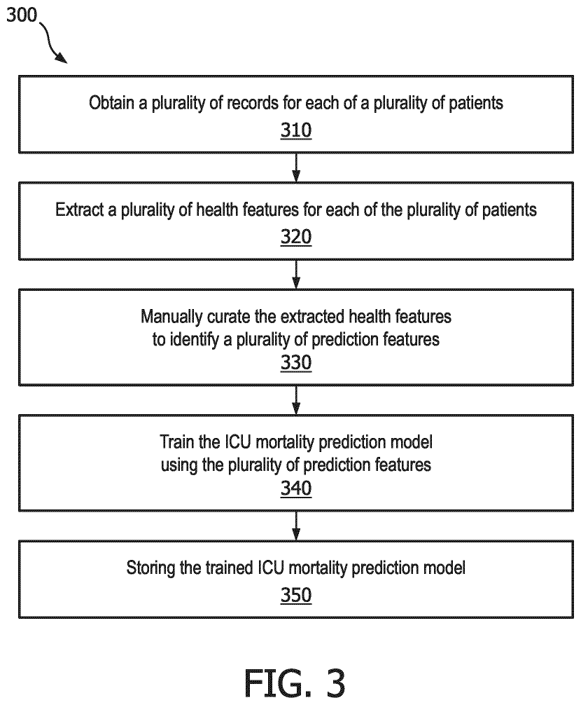

For example, with reference to FIG. 3, a flowchart of a method 300 for training an ICU mortality prediction model is illustrated according to aspects of the present disclosure. In embodiments, the ICU mortality prediction model may be trained by the ICU mortality prediction system 200 and/or may be provided to the ICU mortality prediction system 200 after having already been trained by another similar system.

At a step 310, the method 300 includes obtaining a training dataset 280 comprising a plurality of records for a plurality of patients. In embodiments, the plurality of records for the plurality of patients may be obtained from an electronic medical records database 270B. For example, the electronic medical records database 270B may include patient unit stays admitted to ICUs where physiologic, diagnosis, and treatment information (collectively, “medical information” or “medical data”) are captured, charted, or otherwise recorded. In embodiments, this may be the same electronic medical records database 270A, or may be a different electronic medical records database 270B. In embodiments, the use of the electronic medical records database 270 may be certified as necessary regulatory and privacy standards.

In embodiments, the plurality of records obtained in step 310 can include medical records that cover at least a first period of time for each of the plurality of patients. For example, the plurality of records obtained in step 310 can include medical records that cover the first 24 hours of each patients' stay in an ICU (i.e., the medical data available through the first day of ICU admission). Alternatively, if medical records for one or more of the patients within the first day of ICU admission are not available, the medical records may include, for example and without limitation, medical records covering up to six hours before ICU admission.

As such, in various examples, the first period of time covered by each of the plurality of medical records can include the first 24 hours of a patient's ICU stay, only the first 24 hours of a patient's ICU stay, less than 24 hours of a patient's ICU stay, an amount of time (e.g., 1 hour, 3 hours, 6 hours, 12 hours, etc.) preceding a patient's admission to the ICU, and/or some combination thereof.

At a step 320, the method 300 includes extracting a plurality of health features for each of the plurality of patients from the training dataset 280 obtained in step 310. In embodiments, these health features may be clinical features representing a patient's ICU stay.

For example, continuous features commonly measured (e.g., vital signs, chemistry labs, basic characteristics, etc.) may be included, as well as other continuous features less commonly measured (e.g., lactate, pH, etc.). Health features with many nominal values may be collapsed with cut points defined by clinical knowledge and data distribution to ensure clinically meaningful groups with large enough sample sizes to support stable coefficient estimation.

At a step 330, the method 300 includes curating the extracted plurality of health features in order to identify and define a set of ICU prediction features. That is, the extracted plurality of health features may be curated to identify and define the plurality of different defined ICU prediction features (such as the plurality of defined ICU prediction features using steps 130, 140 of a method 100).

In embodiments, these features may be selected to capture a clinically accurate reflection of the patient while minimizing the impact of potentially anomalous outlier values through the use of means and measures of variability. For example, the primary admission diagnosis strings received as part of the plurality of records may be regrouped based on clinical knowledge to minimize the risk of misclassification. In some embodiments, a defined set of unique ICU admission diagnosis groups may be utilized, whereby unassigned or rare diagnoses are collapsed into a distinct category. Put another way, in some embodiments, the diagnosis upon admission to the ICU is one of the plurality of different defined ICU prediction features and the curation step 330 includes grouping admission diagnoses into one or more unique ICU admission diagnosis groups using clinical knowledge to minimize misclassification.

In particular embodiments, the step 330 can include curating the plurality of different health features extracted in step 320 such that outlier bias is minimized. In further embodiments, the step 330 can include manually curating one or more of the plurality of different health features extracted in step 320.

At a step 340, the method 300 includes training the ICU mortality prediction model using the plurality of different defined ICU prediction features curated in step 330. In embodiments, the ICU mortality prediction model may be trained using a plurality of different defined ICU prediction features corresponding to at least some of the plurality of patients for which medical records were obtained in step 310 (i.e., the training dataset 280).

In embodiments, training the ICU mortality prediction model 264 at step 340 can further include introducing random effects (intercepts and slopes) for vital signs over the admission diagnosis groups to allow vital signs to have different associations with outcomes across diagnosis groups. For example, the random effects may be fitted along with other fixed effects in a generalized linear mixed model. In further examples, the random effects and fixed effects coefficients may be optimized in the penalized iteratively reweighted least square step by assigning points per axis for evaluating adaptive Gauss-Hermite approximates to log-likelihood.

At a step 350, the method 300 includes storing the trained ICU mortality prediction model 264. In embodiments, the trained ICU mortality prediction model 264 may be stored in the memory 260 of an ICU mortality prediction system 200. In other embodiments, the trained ICU mortality prediction model 264 may be stored remotely from an ICU mortality prediction system 200, such as in a remote database accessible by an ICU mortality prediction system 200 (e.g., via communications interface 250 and network 214).

As described herein, the methods and systems of predicting a likelihood of ICU mortality for a patient achieve improved performance over existing approaches, including better performance among subgroups of different admission diagnoses, ICU types, and over different ICUs and years. For example, as discussed below with respect to FIGS. **, the methods and systems of the present disclosure were assessed in relation to two existing models (i.e., APACHE IVa and IVb) using the area under the receiver operating characteristic curve (AUROC), using the actual/predicted ratios, and using subgroups identified by ICU admission diagnosis. Further, the robustness to changes in GCS documentation practice was validated on historic cohorts and compared with APACHE IVa.

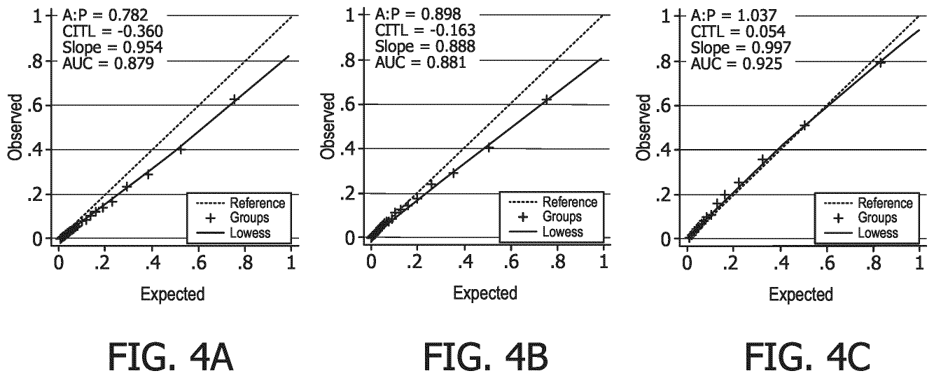

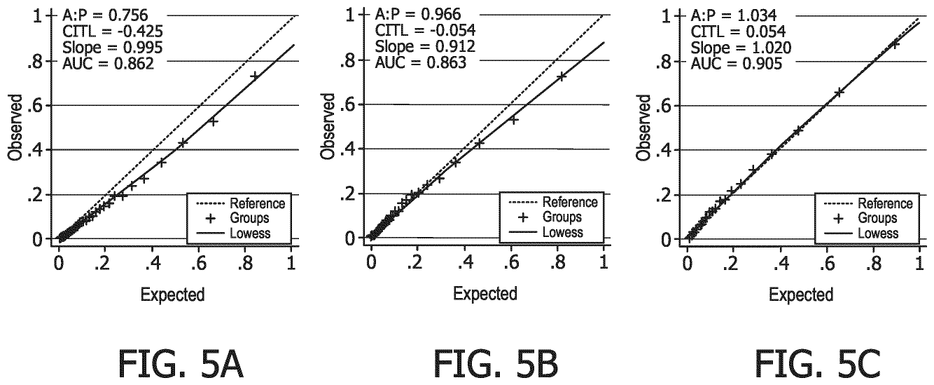

With reference to FIGS. 4A, 4B, and 4C, ICU mortality model performance in a first validation dataset comprising available data spanning 2017-2018 is illustrated. More specifically, FIG. 4A corresponds to APACHE IVa, FIG. 4B corresponds to APACHE IVb, and FIG. 4C corresponds to a trained ICU mortality prediction model 264 of the present disclosure. As shown, the trained ICU mortality prediction model 264 of the present disclosure resulted in higher model discrimination (AUROC) and better calibration (actual/predicted ratios closer to 1; calibration-in-the-large values closer to 0) than the APACHE models. Similar results are also seen in FIGS. 5A, 5B, and 5C, which illustrate hospital mortality model performance in a validation set spanning 2017-2018 using APACHE IVa, APACHE IVb, and the inventive model, respectively.

The improved model performance of the inventive trained ICU mortality prediction model 264 of the present disclosure may also be observed when stratifying analyses by admission diagnosis string. For example, the inventive model performance was evaluated relative to APACHE IVb for a dataset spanning 2014-2019 as shown in Table 2:

TABLE 2
MODEL PERFORMANCE BY ADMISSION
DIAGNOSIS GROUPS

| | ICU Mortality - AUROC | |
|---|---|---|
| Admission Diagnosis Group | APACHE IVb | Inventive Ex. |
| CABG (exclude CABG alone) | 0.741 | 0.898 |
| CABG alone | 0.753 | 0.886 |
| Uncontrolled Hypertension | 0.719 | 0.848 |
| Valve Replacement | 0.812 | 0.926 |
| Transplant | 0.81 | 0.92 |
| CHF | 0.772 | 0.859 |
| Respiratory Arrest | 0.768 | 0.851 |
| Shock Obstructive | 0.829 | 0.911 |
| CV Med | 0.822 | 0.897 |
| Infection Genitourinary | 0.818 | 0.892 |
| GI perforation/rupture | 0.824 | 0.897 |
| Infection Resp | 0.779 | 0.851 |
| Infection GI | 0.832 | 0.903 |
| ARDS | 0.76 | 0.83 |
| Cardiac Arrest | 0.752 | 0.822 |
| Respiratory Surg | 0.841 | 0.909 |
| Infection other | 0.827 | 0.895 |
| Cancer Surg | 0.823 | 0.89 |
| Shock Cardiogenic | 0.78 | 0.845 |
| Infection Subcut | 0.838 | 0.902 |
| Endarterectomy | 0.836 | 0.9 |
| ARF | 0.82 | 0.883 |
| Muscle Skeleton Med | 0.835 | 0.896 |
| Seizures (primary-no | 0.844 | 0.904 |
| structural brain disease) | | |
| Coma | 0.833 | 0.892 |
| GI bleeding | 0.861 | 0.919 |
| Aneurysm | 0.855 | 0.913 |
| Metabolic | 0.859 | 0.916 |
| Rhythm disturbance | 0.859 | 0.916 |
| (atrial, supraventricular) | | |
| Rhythm disturbance (ventricular) | 0.834 | 0.89 |
| Pancreatitis | 0.863 | 0.918 |
| Thoracotomy for lung cancer | 0.82 | 0.875 |
| DKA | 0.901 | 0.956 |
| Overdose/withdraw | 0.893 | 0.948 |
| Respiratory Med | 0.813 | 0.8667 |
| Cardiovascular Surg | 0.88 | 0.931 |
| GI Med | 0.848 | 0.898 |
| Angina Stable | 0.842 | 0.891 |
| ACS | 0.873 | 0.921 |
| Neuro Med | 0.836 | 0.884 |
| Cardiovascular Med | 0.855 | 0.902 |
| Hem Onc Med | 0.85 | 0.895 |
| Infection Neuro | 0.82 | 0.864 |
| GI Surg | 0.881 | 0.924 |
| Trauma | 0.886 | 0.928 |
| Cancer Med | 0.867 | 0.908 |
| Hematoma, subdural | 0.867 | 0.908 |
| Muscle Skeleton Surg | 0.891 | 0.931 |
| Head only trauma | 0.915 | 0.942 |
| CVA | 0.895 | 0.92 |
| Rhythm disturbance | 0.888 | 0.904 |
| (conduction defect) | | |
| Other Med | 0.839 | 0.855 |
| Neuro Surg | 0.925 | 0.939 |
| Asthma | 0.922 | 0.928 |

As shown above, in addition to an increase in mean and median AUROCs, a narrower dispersion of AUROCs across individual diagnosis strings compared to APACHE IVb was observed.

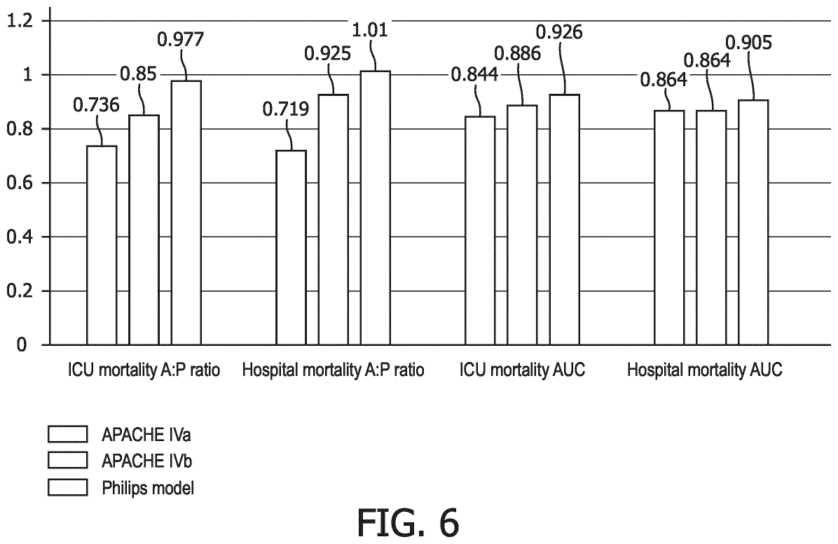

With reference to FIG. 6, the improved model performance of the inventive trained ICU mortality prediction model 264 of the present disclosure was also observed on an extended dataset from 2014-2019 containing a final cohort of over 2 million patient unit stays, where the inventive ICU mortality prediction model 264 outperformed APACHE IVa and IVb in ICU mortality actual/predicted ratio, hospital mortality actual/predicted ratio, ICU mortality AUC, and hospital mortality AUC, as shown in the graph of FIG. 5.

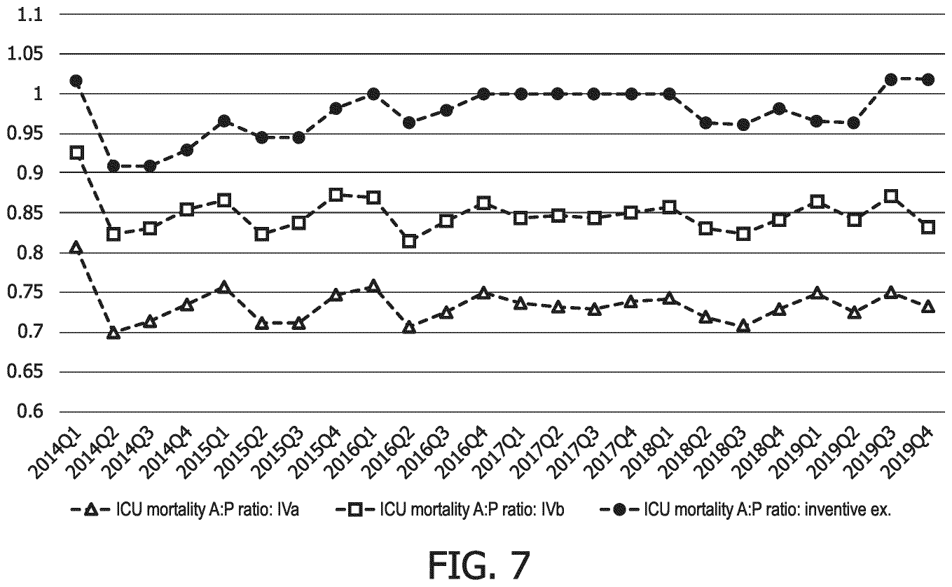

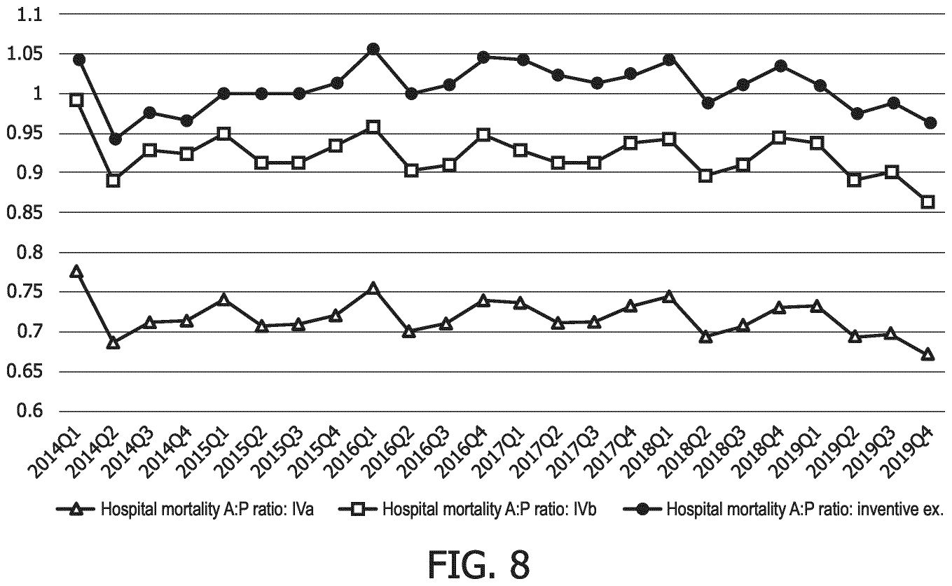

As shown in FIG. 7 and FIG. 8, consistently improved model performance of the inventive trained ICU mortality prediction model 264 of the present disclosure was also observed on the extended dataset over several years.

Additionally, as shown in Table 3 below, the improved model performance of the inventive trained ICU mortality prediction model 264 of the present disclosure was also observed across different ICU types:

TABLE 3
MODEL PERFORMANCE BY ICU TYPE

| | ICU mortality: AUROC | | | Hospital mortality: AUROC | | |
|---|---|---|---|---|---|---|
| | | | Inventive | | | Inventive |
| Unit types | IVa | IVb | Ex. | IVa | IVb | Ex. |
| Burn-Trauma | 0.867 | 0.871 | 0.95 | 0.876 | 0.859 | 0.925 |
| ICU | | | | | | |
| CCU-CTICU | 0.89 | 0.891 | 0.931 | 0.874 | 0.873 | 0.914 |
| CSICU | 0.885 | 0.886 | 0.945 | 0.866 | 0.865 | 0.919 |
| CTICU | 0.88 | 0.879 | 0.928 | 0.866 | 0.862 | 0.907 |
| Cardiac ICU | 0.892 | 0.892 | 0.931 | 0.872 | 0.872 | 0.911 |
| MICU | 0.869 | 0.87 | 0.912 | 0.851 | 0.851 | 0.893 |
| Med-Surg ICU | 0.883 | 0.884 | 0.926 | 0.86 | 0.86 | 0.902 |
| Neuro ICU | 0.898 | 0.902 | 0.932 | 0.877 | 0.879 | 0.915 |
| SICU | 0.891 | 0.891 | 0.929 | 0.867 | 0.865 | 0.907 |
| Trauma ICU | 0.899 | 0.896 | 0.935 | 0.888 | 0.883 | 0.92 |
| Vascular ICU | 0.923 | 0.918 | 0.937 | 0.888 | 0.89 | 0.898 |

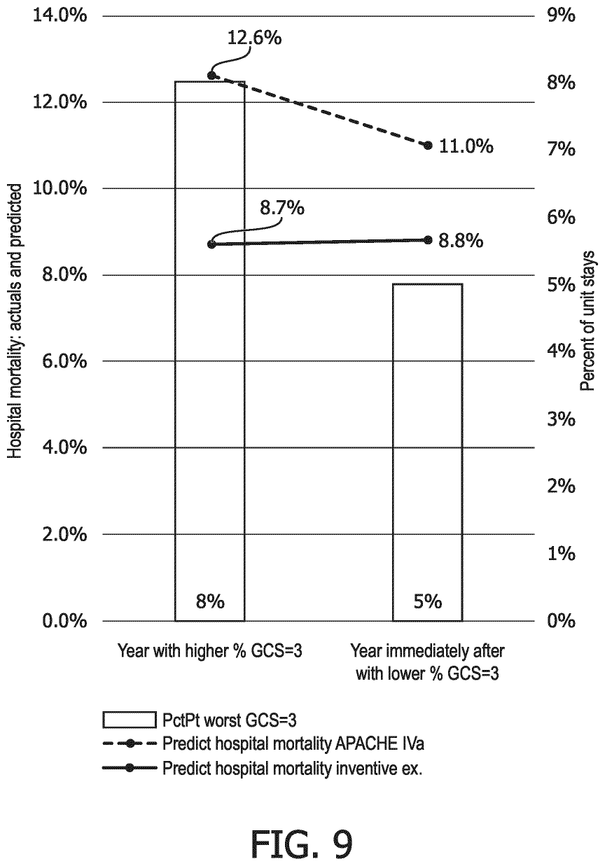

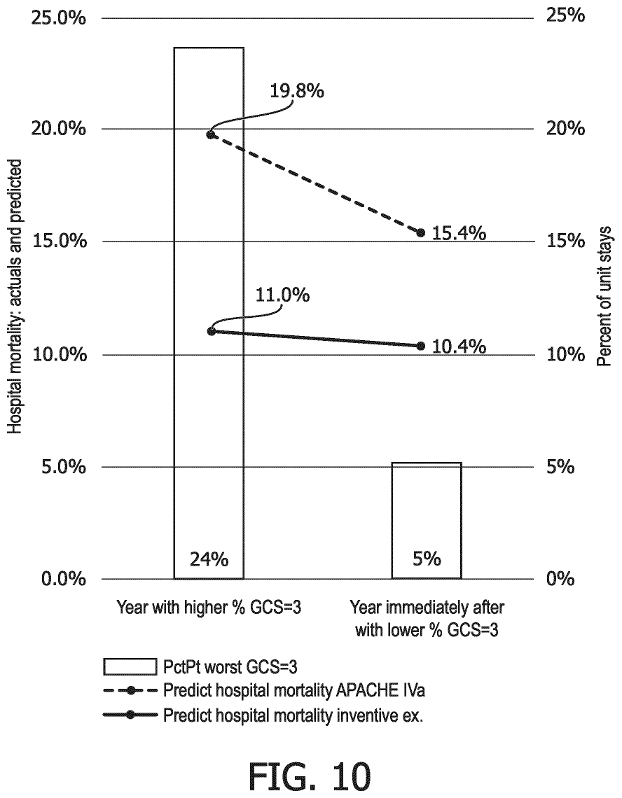

With reference to FIG. 9 and FIG. 10, the effect of changes in GCS documentation patterns is illustrated for two health systems (representing over 25 ICUs) that previously experienced a substantial change in APACH-adjusted mortality performance after changes in GCS documentation practice. As shown, there was a significant change in the proportion of GCS scores equal to three before and after the GCS documentation pattern change, confirming the impact of the documentation change on GCS scores.

For example, as shown in FIG. 9, a change in predicted mortality after an inadvertent change in GCS documentation practice in two consecutive years is illustrated. Further, as shown in FIG. 10, a change in predicted mortality after a deliberate change in GCS documentation practice in two consecutive years is illustrated. In both cases, a significant difference in the predicted mortality using the existing APACHE IVa model was observed when compared with a trained model of the present disclosure, which shows that the inventive models are less susceptible to biases due to changes in documentation practices.

According to an embodiment, the ICU mortality prediction is configured to process many thousands or millions of datapoints to extract the plurality of different defined ICU prediction features for a patient, to generate the likelihood of ICU mortality for the patient, and to display the likelihood of ICU mortality for the patient to a user via the user interface. Further, preferably data for 100s or 1000s of patients are used to train the ICU mortality prediction model 264. Accordingly, the ICU mortality prediction system is configured to process millions of datapoints to extract the plurality of different defined ICU prediction features for these 100s or 1000s of patients and use that data to train the ICU mortality prediction model 264. This requires millions or billions of calculations, which a human mind could not perform in a lifetime. Further, since training the ICU mortality prediction model 264 utilizes a unique data set, the stored trained ICU mortality prediction model is a novel model.

By providing improved prediction of the likelihood of ICU mortality for a patient, this novel ICU mortality prediction system has an enormous positive effect on patient care compared to prior art systems. Improved understanding of the likelihood of ICU mortality for a patient can improve patient care and health, and prioritize care and resources, thereby saving lives of ICU patients as well as all patients within a care facility.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

As used herein, although the terms first, second, third, etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

Unless otherwise noted, when an element or component is said to be “connected to,” “coupled to,” or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

It should also be understood that, unless clearly indicated to the contrary, in any methods claimed herein that include more than one step or act, the order of the steps or acts of the method is not necessarily limited to the order in which the steps or acts of the method are recited.

The above-described examples of the described subject matter can be implemented in any of numerous ways. For example, some aspects can be implemented using hardware, software or a combination thereof. When any aspect is implemented at least in part in software, the software code can be executed on any suitable processor or collection of processors, whether provided in a single device or computer or distributed among multiple devices/computers.

The present disclosure can be implemented as a system, a method, and/or a computer program product at any possible technical detail level of integration. The computer program product can include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium comprises the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, comprising an object oriented programming language such as Smalltalk, C++, or the like, and procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, comprising a local area network (LAN) or a wide area network (WAN), or the connection can be made to an external computer (for example, through the Internet using an Internet Service Provider). In some examples, electronic circuitry comprising, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to examples of the disclosure. It will be understood that each block of the flowchart illustrations and/or block diagrams, and combinations of blocks in the flowchart illustrations and/or block diagrams, can be implemented by computer readable program instructions.

The computer readable program instructions can be provided to a processor of a, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and/or other devices to function in a particular manner, such that the computer readable storage medium having instructions stored therein comprises an article of manufacture comprising instructions which implement aspects of the function/act specified in the flowchart and/or block diagram or blocks.

The computer readable program instructions can also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various examples of the present disclosure. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Other implementations are within the scope of the following claims and other claims to which the applicant can be entitled.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A method for predicting a likelihood of intensive care unit (ICU) mortality for a patient, the method comprising:
obtaining, from an electronic medical records database, a plurality of records for a patient in an ICU covering at least a first time period;
extracting, from the obtained plurality of records, a plurality of different defined ICU prediction features for the patient;
analyzing the extracted plurality of different defined ICU prediction features using a trained ICU mortality prediction model; and
predicting, by the trained ICU mortality prediction model, a likelihood of ICU mortality for the patient.

2. The method of claim 1, wherein the first time period is at least 24 hours in the ICU.

3. The method of claim 1, wherein the extracted plurality of different defined ICU prediction features for the subject comprises one or more of BMI; age; gender; pre-ICU admission lead time; ventilation status at hour 24 of ICU admission; whether the subject was admitted with elective surgery status; mean blood pressure; systolic blood pressure; diastolic blood pressure; heart rate; respiratory rate; oxygen saturation; blood glucose; white blood cell count; blood sodium; blood potassium; blood creatinine; blood hemoglobin; blood albumin; blood lactate; arterial blood gas, pH; arterial blood gas, PaCO2; admission diagnosis; and Total Glasgow Coma Scale score.

4. The method of claim 1, wherein the likelihood of ICU mortality for the patient further comprises a mortality timeline.

5. The method of claim 1, wherein the predicting of the likelihood of ICU mortality is performed by a ICU mortality prediction system that is a component of a patient data management systems (PDMS) or a patient monitoring system.

6. The method of claim 1, wherein the patient is a historical patient.

7. The method of claim 1, wherein the patient is in the ICU during the presentation of the generated likelihood of ICU mortality.

8. The method of claim 1, wherein the trained ICU mortality prediction model is configured to analyze the extracted plurality of different defined ICU prediction features and predict the likelihood of ICU mortality for the patient when some of the plurality of different defined ICU prediction features are missing from the obtained plurality of records.

9. The method of claim 1, wherein the extracted plurality of different defined ICU prediction features comprises a total Glasgow Coma Scale score (GCS), wherein the GCS is assessed most recently during the first time period.

10. The method of claim 1, wherein the ICU mortality prediction model is a generalized additive model (GAM).

11. An intensive care unit (ICU) mortality prediction system configured to predict a likelihood of ICU mortality for a patient, the system comprising:
an electronic medical records database comprising a plurality of records for a plurality of patients; and
a processor configured to:
(i) obtain, from the electronic medical records database, a plurality of records for the patient in an ICU covering at least a first time period;
(ii) extract, from the obtained plurality of records, a plurality of different defined ICU prediction features for the patient;
(iii) analyze the extracted plurality of different defined ICU prediction features using a trained ICU mortality prediction model; and
(iv) predict, by the trained ICU mortality prediction model, a likelihood of ICU mortality for the patient.

12. The system of claim 11, wherein the first time period is at least 24 hours in the ICU.

13. The system of claim 11, wherein the extracted plurality of different defined ICU prediction features comprises one or more of BMI; age; gender; pre-ICU admission lead time; ventilation status at hour 24 of ICU admission; whether the subject was admitted with elective surgery status; mean blood pressure; systolic blood pressure; diastolic blood pressure; heart rate; respiratory rate; oxygen saturation; blood glucose; white blood cell count; blood sodium; blood potassium; blood creatinine; blood hemoglobin; blood albumin; blood lactate; arterial blood gas, pH; arterial blood gas, PaCO2; admission diagnosis; and Total Glasgow Coma Scale score.

14. The system of claim 11, wherein the ICU mortality prediction system is a component of a patient data management systems (PDMS) or a patient monitoring system.

15. The system of claim 11, wherein the ICU mortality prediction model is a generalized additive model (GAM).

16. The method of claim 1, wherein the ICU mortality prediction model is trained by:
obtaining, from an electronic medical records database, a plurality of historical records for each of a plurality of historical patients in an ICU;
extracting, from the obtained plurality of historical records, a plurality of different health features for each of the plurality of historical patients;
curating the extracted plurality of different health features to identify a plurality of different historical ICU prediction features, wherein a duration is configured to minimize outlier bias, and wherein admission diagnosis is one of the plurality of different historical ICU prediction features and further wherein curation comprises grouping admission diagnoses into one or more groups using clinical knowledge to minimize misclassification;
training the ICU mortality prediction model using the plurality of different historical ICU prediction features; and
storing the trained ICU mortality prediction model.

17. The method of claim 1, further comprising:
presenting, via a user interface, the predicted likelihood of ICU mortality for the patient.

18. The system of claim 11, wherein the ICU mortality prediction model is trained by:
obtaining, from an electronic medical records database, a plurality of historical records for each of a plurality of historical patients in an ICU;
extracting, from the obtained plurality of historical records, a plurality of different health features for each of the plurality of historical patients;
curating the extracted plurality of different health features to identify a plurality of different historical ICU prediction features, wherein the curation is configured to minimize outlier bias, and wherein admission diagnosis is one of the plurality of different historical ICU prediction features and further wherein curation comprises grouping admission diagnoses into one or more groups using clinical knowledge to minimize misclassification;
training the ICU mortality prediction model using the plurality of different historical ICU prediction features; and
storing the trained ICU mortality prediction model.

19. The system of claim 18, wherein:
the different historical ICU prediction features include vital signs, and
the ICU mortality prediction model is further trained by introducing random intercepts and slopes for vital signs over the admission diagnosis groups.

20. The system of claim 11, further comprising:
a user interface configured to provide the predicted likelihood of ICU mortality.
